(19) United States
(12) Patent Application Publication
(10) Pub. No.: US 2008/0086797 A1
(43) Pub. Date: Apr. 17, 2008

(54) Mask frame

(75) Inventor: Chih-Cheng Shiue, Escondido, CA (US)

Correspondence Address: BRUCE H. TROXELL, 5205 LEESBURG PIKE, SUITE 1404, FALLS CHURCH, VA, 22041, US

(73) Assignee: QDS Injection Molding LLC.

(21) Appl. No.: 11/580,299

(22) Filed: Oct. 13, 2006

(51) Int. Cl. A61F 9/04

(52) U.S. Cl. 2/440

(57) ABSTRACT

A mask is disclosed. In one embodiment, the mask is a diving mask and includes a soft flirt; a plastic frame formed with the flirt; a strap interconnected both ends of the frame; two auxiliary eye frames; two lenses fitted in the frame by securing the auxiliary eye frames to the frame; and a brace either fully or partially formed in either a central portion of the frame or a connecting portion of the auxiliary eye frames by subjecting to injection molding and cooling processes. The brace includes holes, latches, and recesses for increasing its secure engagement with the frame. The enhanced frame can effectively resist collision.

### BACKGROUND OF THE INVENTION

1. Field of Invention

The invention relates to masks for diving, sports, or industry, and more particularly to such a mask (e.g., diving mask) having an enhanced frame by integrally forming a brace therewith.

2. Related Art

Conventional diving masks or other types of mask are mounted with a frame made of plastic. Also, a metal brace is mounted in the frame for increasing its structural strength. The brace is typically mounted by means of mated holes and projections, auxiliary eye frames, or screws. However, such assembly is not reliable when a strong external force is suddenly applied to the frame. The brace may even detach from the frame in a worst case.

An exploded view of a conventional diving mask incorporating the above components is shown in FIG. 1. The mask comprises a soft flirt A, two lenses B, a frame D, a strap (not shown) interconnected both ends of the frame D, two auxiliary eye frames E, and a metal brace C mounted on a central portion (i.e., bridge) of the frame D by sandwiching the brace C between the auxiliary eye frames E and the frame D.

However, the prior art diving mask is not durable. For example, the brace C may be displaced in the frame D when a strong external force is suddenly applied to the mask. To the worse, the mask may be broken. As a result, water may enter the mask while diving.

The frame D is the main component for securely fitting the lenses B thereon. Thus, a structurally strong frame D is essential to the construction of a durable mask.

Further, the brace C is an aesthetic member in constructing a mask. Thus, the need for improvement of the brace also exists in order to contribute significantly to the advancement of the art.

### SUMMARY OF THE INVENTION

It is therefore an object of the present invention to provide a mask having an enhanced frame for effectively resisting collision.

It is another object of the present invention to provide a mask having a hard brace integrally formed in a central portion of the frame by subjecting to injection molding and cooling processes.

It is yet another object of the present invention to provide a mask having a brace including one or more holes, one or more latches, and one or more recesses wherein liquid plastic is poured either into the holes and the recesses or around the latches so as to securely form the frame and the brace as a whole after cooling.

It is a further object of the present invention to provide a mask having two auxiliary eye frames and a brace formed in a connecting portion of the auxiliary eye frames by subjecting to injection molding and cooling processes.

In one aspect of the present invention the either is fully or partially disposed in either a central portion of the frame or a connecting portion of the auxiliary eye frames.

In another aspect of the present invention the brace is formed of metal.

The above and other objects, features and advantages of the present invention will become apparent from the following detailed description taken with the accompanying drawings.

### BRIEF DESCRIPTION OF THE DRAWINGS

FIG. 1 is an exploded view of a conventional diving mask;

FIG. 2 is an exploded view of a first preferred embodiment of diving mask according to the present invention;

FIG. 3 is a perspective view of the brace of FIG. 2;

FIG. 4 is a longitudinal sectional view of a central portion of the frame with the brace formed therein; and

FIG. 5 is an exploded view of a second preferred embodiment of diving mask according to the present invention.

### DETAILED DESCRIPTION OF THE INVENTION

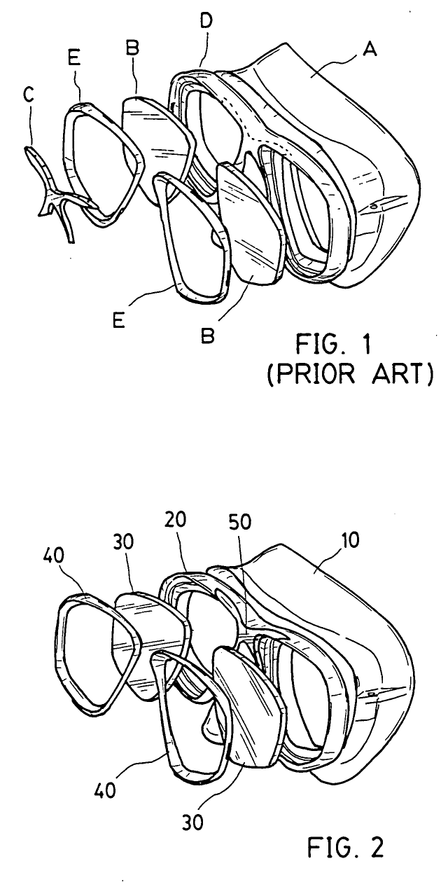

Referring to FIG. 2, a diving mask in accordance with a first preferred embodiment of the present invention is shown. While the diving mask is taken as an exemplary example of the invention throughout the description, it is understood that the invention also applies to other types of mask (e.g., breathing masks, sports masks, etc.).

The mask comprises a soft flirt 10, a pair of lenses 30, a frame 20, a strap (not shown) interconnected both ends of the frame 20, a pair of auxiliary eye frames 40, and a brace 50. The flirt 10, the lenses 30, and the auxiliary eye frames 40 are well known devices. Accordingly, further description thereof is omitted for purposes of brevity and convenience only, and is not limiting.

The brace 50 is made of a material (e.g., metal or the like) harder than that of the frame 20 which is made of plastic as detailed later.

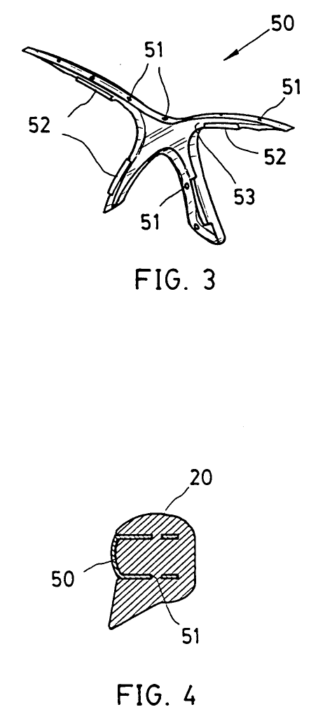

Referring to FIG. 3, the brace 50 is shaped to be formed with the frame 20 of the invention. Note that the brace 50 may be shaped other than above in other embodiments without departing from the scope of the invention. The brace 50 is formed on a central portion (i.e., bridge) of the frame 20. The brace 50 comprises a plurality of holes 51, a plurality of latches 52, and two opposite nose recesses 53. In a manufacturing process (e.g., injection molding) of the frame 20, liquid plastic is poured either into the holes 51 and the recesses 53 or around the latches 52. Thus, the frame 20 and the brace 50 can be formed as a whole after cooling.

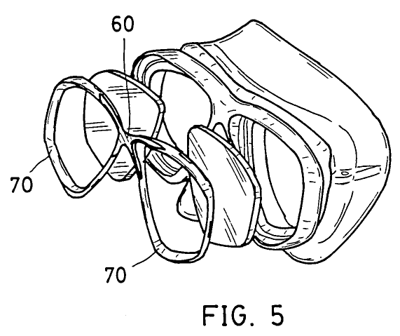

Referring to FIG. 4, it shows the integrally formed frame 20 and the brace 50. As shown, the holes 51 are filled with plastic material which forms the frame 20. Also, the brace 50 can be either fully formed in the frame 20 or partially formed therein.

Referring to FIG. 5, a second preferred embodiment of diving mask according to the present invention is shown. The brace 60 is formed in a connecting portion (e.g., bridge) of a pair of auxiliary eye frames 70. Likewise, the brace 60 and the auxiliary eye frames 70 are formed as a whole after subjecting to injection molding and cooling processes.

In brief, either (i) the brace 50 is integrally formed with the frame 20 in the first preferred embodiment or (ii) the brace 60 is integrally formed with the auxiliary eye frames 70 in the second preferred embodiment after subjecting to injection molding and cooling processes. As a result, the structural strength of the mask is greatly increased.

It is to be understood that the present invention is by no means limited only to the particular constructions herein disclosed and shown in the drawings, but also comprises any modifications or equivalents within the scope of the claims.

What is claimed is:

1. A mask comprising:
a soft flirt;
a plastic frame formed with the flirt;
a strap interconnected both ends of the frame;
a pair of auxiliary eye frames;
a pair of lenses fitted in the frame by securing the auxiliary eye frames to the frame; and
a brace formed of a material harder than that of the frame and either fully or partially disposed in either a central portion of the frame or a connecting portion of the auxiliary eye frames.

2. The mask of claim 1, wherein the brace is integrally formed with either the frame or the auxiliary eye frames by subjecting to injection molding and cooling processes.

3. The mask of claim 1, wherein the material of forming the brace is metal.

4. The mask of claim 1, wherein the brace comprises at least one of (i) one or more holes, (ii) one or more latches, and (iii) one or more recesses.
